(19) United States
(12) Patent Application Publication
(10) Pub. No.: US 2009/0054820 A1
(43) Pub. Date: Feb. 26, 2009

(54) STATIC PROGRESSIVE PRONATION SUPINATION SPLINT

(76) Inventors: Thomas R. Weltner, San Luis Obispo, CA (US); Richard D. Curley, JR., Atascadero, CA (US); Mark T. Ingersoll, Atascadero, CA (US)

Correspondence Address: LUEDEKA, NEELY & GRAHAM, P.C., P O BOX 1871, KNOXVILLE, TN, 37901, US

(21) Appl. No.: 12/037,998

(22) Filed: Feb. 27, 2008

Related U.S. Application Data
(60) Provisional application No. 60/891,981, filed on Feb. 28, 2007.

(51) Int. Cl. A61F 5/00

(52) U.S. Cl. 602/21

(57) ABSTRACT

A patient care system for providing static progressive positioning of body members in pronation or supination, the system includes a positioning assembly that rotatably positions a first body engaging assembly relative to a second body engaging assembly for static progressive positioning of a body joint in pronation or supination.

### CROSS-REFERENCE TO RELATED APPLICATION

This application claims priority to United States Provisional Application Ser. No. 60/891,981, filed Feb. 28, 2007, and entitled STATIC PROGRESSIVE PRONATION SUPINATION SPLINT, and incorporated by reference herein in its entirety.

### FIELD

This disclosure relates generally to orthopedic treatment devices. More particularly, the disclosure relates to orthosis devices for treating joint stiffness and soft tissue contractures.

### BACKGROUND

In the treatment of joint stiffness or contractures which decrease the range of motion of the joint, an orthotic device may be used to urge the joint to a desired pronation or supination orientation and maintain the orientation for a prescribed period of time. For example, to treat a wrist, it may be desirable to urge the wrist towards the limits of its current range of movement. Such treatments are generally referred to as static-progressive treatments and are used to elongate the soft tissue over time. Improvement is desired in the construction of devices for providing static progressive treatment of joints and for moving the wrist from supine to prone positions.

The disclosure relates to patient care systems, such as orthotic devices, of improved construction.

### SUMMARY

The patient care systems described herein are configured for providing static progressive positioning of body members in pronation or supination. The systems are useful for managing contractures due to neurological disorders, muscle hypertonicity or orthopedic conditions affecting the body member. The systems may be utilized to increase the range of motion. The systems are also suitable for post-operative use to limit or prevent rotation and other movements of the body member.

In one embodiment, the system includes a first body engaging assembly for engaging first body portions of a user adjacent a joint of the body; a second body engaging assembly for engaging second body portions of the user; and a positioning assembly that rotatably positions the first body engaging assembly relative to the second body engaging assembly for static progressive positioning of the joint in pronation or supination.

The positioning assembly includes a ring member, the first body engaging member being mounted adjacent an interior portion of the ring member, a yoke to which the ring member is movably mounted, and a rotator mounted to the yoke and positioned to rotationally and frictionally engage the ring member to move the ring member for positioning of the joint.

In another aspect, a patient care system is provided having a rotatable ring member; a rotator operably associated with the ring member to rotate the ring member for positioning of the joint; a first body engaging assembly mounted on the ring member and configured for engaging first body portions of a user adjacent a joint of the body; and a second body engaging assembly remote from the first body engaging member and configured for engaging second body portions of the user.

Operation of the rotator causes rotation of the ring member to rotatably position the first body engaging assembly relative to the second body engaging assembly for static progressive positioning of the joint in pronation or supination.

The systems described herein enable the wrist or other joint of a user to be moved through a range of motion. For example, when used with the wrist, the system may be adjusted to position the wrist at any position between pronation and supination, as well as positions beyond.

### BRIEF DESCRIPTION OF THE DRAWINGS

Further features of the disclosure are apparent by reference to the detailed description when considered in conjunction with the figures, which are not to scale so as to more clearly show the details, wherein like reference numbers indicate like elements throughout the several views, and wherein:

FIG. 1 is a perspective view of a wrist orthosis in accordance with a preferred embodiment.

FIG. 2 is a partially exploded perspective view of the wrist orthosis of FIG. 1.

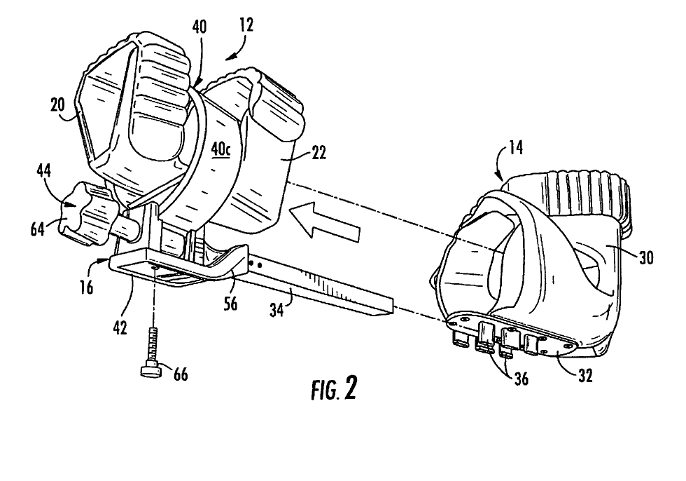

FIG. 3 is an enlarged view of a positioning assembly portion of the orthosis of FIG. 1.

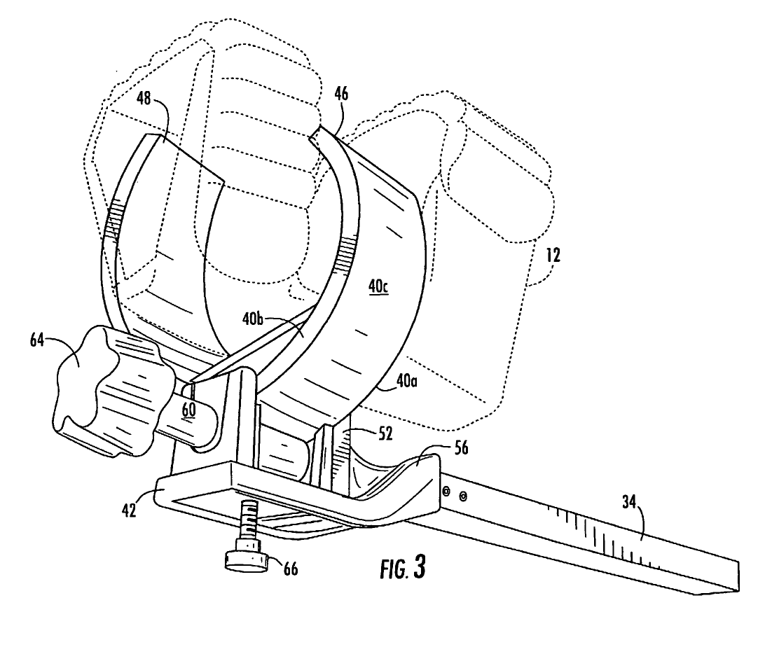

FIG. 4 is an exploded view of components of the positioning assembly portion of FIG. 3.

FIG. 5 is a front end view of the orthosis of FIG. 1, with the orthosis adjusted to position the wrist of a user in supination.

FIG. 6 is a front end view of the orthosis of FIG. 1, with the orthosis adjusted to position the wrist of a user in pronation.

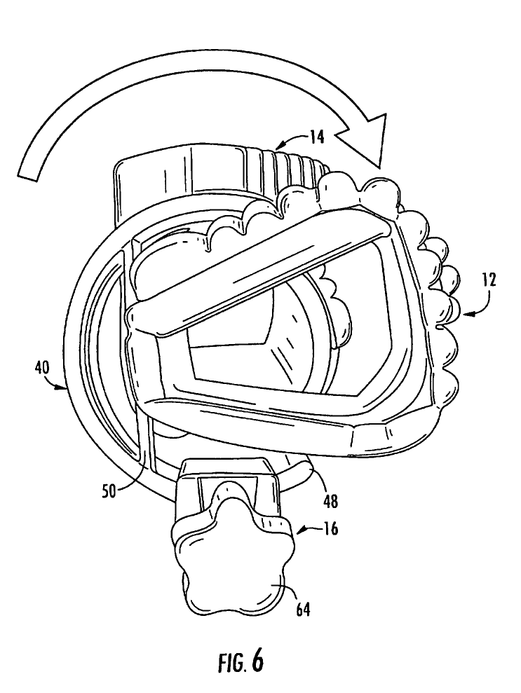

### DETAILED DESCRIPTION

The disclosure relates to devices for static progressive pronation and supination of a body member. The preferred embodiment is described in connection with a wrist orthosis. In this regard, it will be understood that sizes and fit features of the splint may be modified for use with other body members typically treated using static progressive pronation and supination techniques, such as the knee, elbow and ankle.

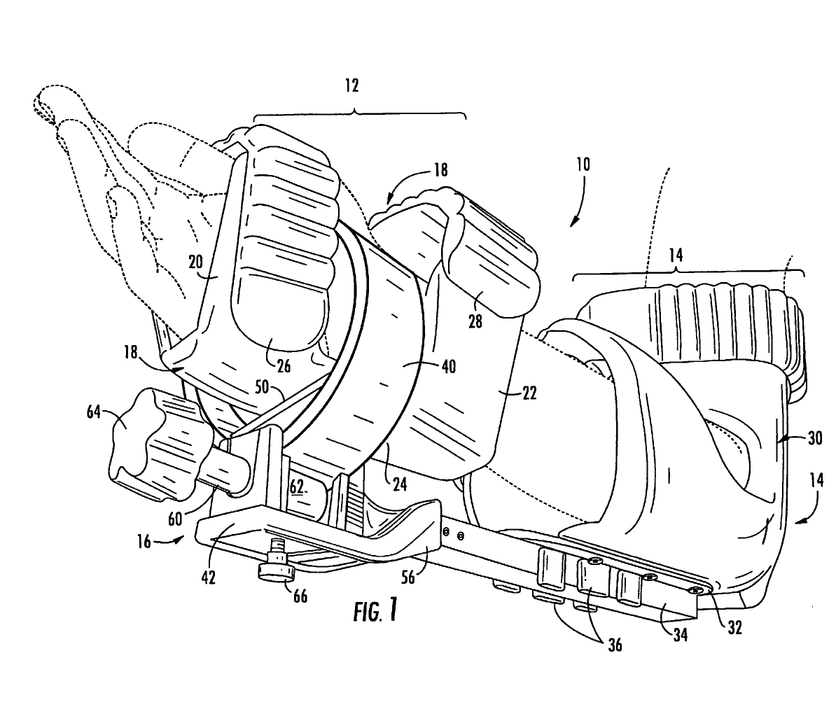

With reference to the drawings, one embodiment of the disclosure relates to a wrist orthosis 10 preferably having a hand/wrist engaging assembly 12, an elbow/arm engaging assembly 14, and a positioning assembly 16 that rotatably positions the hand/wrist engaging assembly 12 relative to the elbow/arm engaging assembly 14 and can be used for static progressive positioning of the wrist joint in pronation or supination or other orientation. As seen in FIG. 1, an arm A of a user is shown in phantom, with the wrist thereof in an intermediate orientation between prone (facing down) and supine (facing up).

The hand/wrist engaging assembly 12 preferably includes a substantially rigid hand/wrist receiving member 18 configured to receive the hand and the wrist of the user. The member 18 includes a substantially V-shaped hand receiver 20 and an aligned and substantially V-shaped wrist receiver 22. The receivers 20 and 22 are spaced apart from one another by a connector segment 24 of the receiver 18. Straps 26 and 28 preferably overlie the open upper ends of the receivers 20 and 22, respectively, and are releasably securable to the receivers 20 and 22 as by use of mating hook and loop material or the like.

The member 18 may be made of a rigid plastic material, such as a high density polyethylene, or a lightweight aluminum material, encased within a flexible semi-rigid material, such as a polyurethane foam. The segment 24 is configured to include bosses or other structure, accessible via apertures for receiving fasteners such as screws for mounting of the member 18 to the positioning assembly 16. The straps 26 and 28 are preferably made of a soft flexible fabric or plastic material overmolded with polyurethane foam or the like. It will be understood, however, that the member 18 and the components thereof may be made in various configurations for engaging and retaining the hand/wrist of the user and may be made of a variety of materials.

The elbow/arm engaging assembly 14 is constructed of similar materials to the assembly 12 and includes a substantially L-shaped open-ended elbow/ann receiver 30 configured for receiving the elbow and portions of the bicep and forearm of the user adjacent the elbow. In this regard, the receiver 30 also preferably includes straps corresponding to the straps 26 and 28 for securing the receiver 30 snugly to the user. A mounting plate 32 is secured to a lower surface of the receiver 30, as by screws or other fasteners.

The mounting plate 32 is configured to be adjustably mounted to an extension member 34 which extends from the positioning assembly 16 to enable the position of the assembly 14 to be adjusted relative to the position of the assembly 12 for fitting the orthosis 10 to different arm lengths. For example, the extension member 34 may be a square aluminum tubing and the mounting plate 32 includes a plurality of spaced apart ribs 36 or other receiving structure configured to fittingly receive the member 34. In this regard, it will be understood that adjustment of the positions of the assemblies 12 and 14 along the aligned longitudinal axes thereof is desired, but that it is desired that the assemblies 12 and 14 remain aligned during use of the orthosis 10.

The positioning assembly 16 is configured to rotatably position the hand/wrist engaging assembly 12 relative to the elbow/arm engaging assembly 14 and can be used for static progressive positioning of the wrist joint in pronation or supination or other orientation. The positioning assembly 16 includes a ring member 40, a yoke 42, and a rotator 44.

The ring member 40 is a partial ring configuration having free ends 46 and 48. The ring member 40 is substantially rigid and may be of one-piece molded plastic construction if desired. The ring member 40 preferably has a diameter of from about 4 to about 5 inches for use with adults. A portion of the ring member 40 is open such that the spacing between the ends 46 and 48 is sufficient to enable a user's wrist to fit through the open space, such as from about 2½ to about 3½ inches. The ring member 40 may also be a complete ring. Also, the ring member 40 may have other than a circular cross-section, and may be other shapes, such as oval, U-shaped, D-shaped, and V-shaped, with the term “Ring member” referring to structure having a generally encircling or partially encircling configuration.

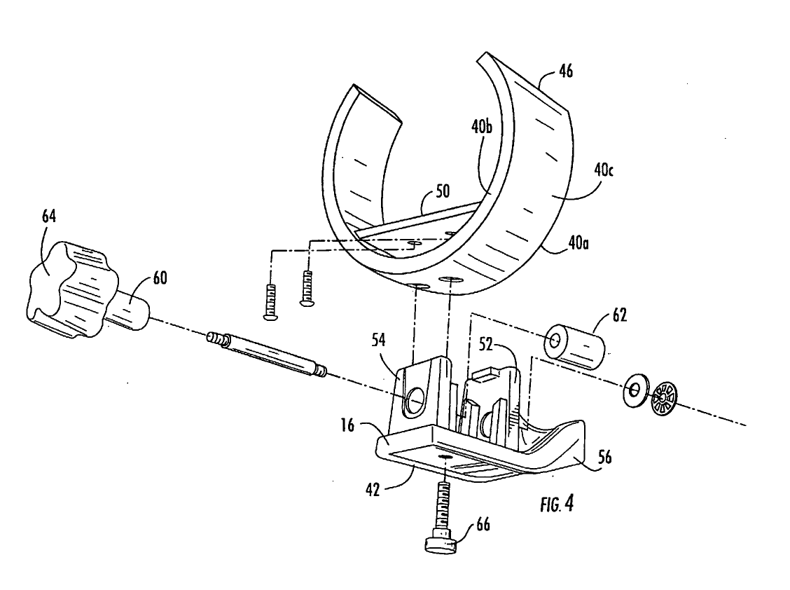

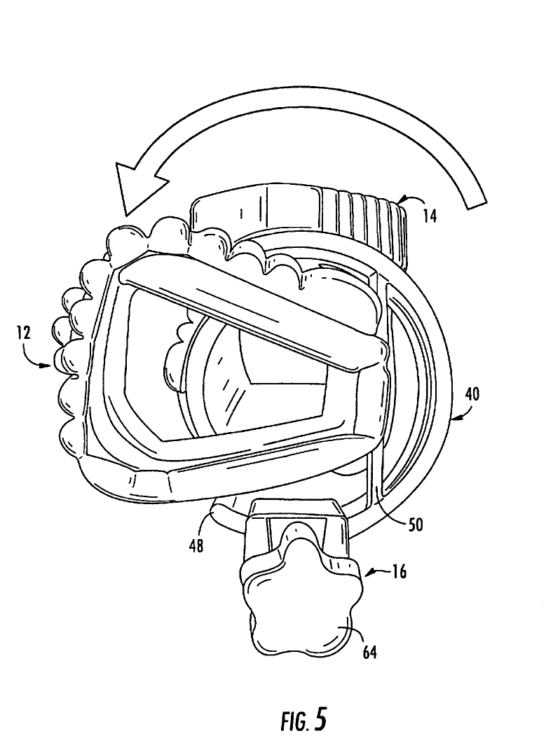

As best seen in FIGS. 4 and 5, a support 50 extends across a portion of the interior of the ring member 40 for mounting of the segment 24 of the hand/wrist receiving member 18. For example, the segment 24 may be secured to the support 50 as by fasteners and the like.

The yoke 42 movably mounts the ring member 40 so that the ring member 40 may be positioned as desired using the positioning assembly 16. For example, the yoke 42 may include spaced apart and mutually facing uprights 52 and 54 having opposed receivers on their interior surfaces, such as open-ended channels, ribs, or the like, for slidably receiving edge portions 40a and 40b of the ring member 40. If desired, ribs may project from the edges 40a and 40b to engage corresponding structure on the uprights or other portion of the yoke 42.

Alternatively, the edge portions 40a and 40b may define grooves for being slidingly received by projections located on the uprights 52 and 54 or other opposed portions of the yoke 42 to movably guide the ring member 40 for positioning of the wrist as described herein. The yoke 42 may include a mount 56 configured for mounting of the extension 34. The mount 56 may be co-molded with the yoke 42 and having a surface shaped to receive a free end of the extension 34. The extension 34 may be secured to the mount 56 as by friction, mechanical fasteners, adhesives, and the like.

The rotator 44 rotationally and frictionally engages the ring member 40 to move the ring member 40 for positioning of the wrist as described herein. The rotator 44 may be rotatably mounted to the yoke 42 and positioned to engage the ring member 40. For example, the rotator may include a shaft 60 that extends though aligned bores of the uprights 52 and 54 so as to be rotatable relative to the yoke 42. A portion of the shaft 60 that extends between the uprights 52 and 54 may include an outer surface, such as a rubber cylinder 62, a coating having a relatively high coefficient of friction, or like structure, for frictionally contacting a contact surface, such as the outer periphery 40c of the ring member 40. In this regard, it will be understood that the outer periphery 40c corresponds to the general encircling or partially encircling shape of the ring member 40.

In this regard, the shaft 60 is located so that the outer surface of the shaft 60, such as the rubber cylinder 62, bears against the contact surface of the ring member 40, such as the outer periphery 40c. When the shaft 60 is rotated, it will be understood that such rotation is transferred, preferably directly, to the ring member 40. In this regard, the distal end of the shaft 60 is rotatably mounted relative to the yoke 42 and the opposite free end of the shaft 60 preferably includes a knob 64 or the like to facilitate manual rotation of the shaft 60.

A stop 66, such as a set screw threadably received by the yoke 42, or the like may be provided to positively engage the shaft 60 or portions thereof, such as the cylinder 62, to positively lock the shaft 60 from rotation. This may be desired, for example, once the ring member 40 has been rotated to desirably position the wrist of a user for treatment and it is desired to retain the wrist in the set orientation for a period of time.

With reference to FIGS. 4-5, it will be seen that the positioning assembly 16 advantageously enables the wrist of a user to be moved through a range of motion. For example, in FIG. 4, the orthosis is adjusted to position the wrist of a user in supination. In FIG. 5, the orthosis is adjusted to position the wrist of a user in pronation.

As will be appreciated, the orthosis may be adjusted to position the wrist at any position between pronation and supination, as well as positions beyond, as permitted by the size and configuration of the ring member 40, as well as the mobility of the user's wrist.

For example, assuming suitable wrist mobility, the ring member 40 may be rotated by the positioning system 16 from a position corresponding to the position of the free end 46 of the ring member 40 adjacent the cylinder 62 to a position corresponding to the position of the free end 48 of the ring member 40 adjacent the cylinder 62. The locations of the free ends 46 and 48 are thus preferably selected to limit the rotation so as to not permit a wrist to be over pronated or supinated beyond the physical limits of wrists.

As mentioned previously, the ring member 40 may also be a complete ring (no free ends). However, in such case it is preferred to have stops, such as projections or the like extending from the periphery 40c at locations corresponding to the locations of the free ends 46 and 48, to limit the extent of rotation.

The foregoing description of preferred embodiments for this disclosure has been presented for purposes of illustration and description. It is not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. A patient care system for providing static progressive positioning of body members in pronation or supination, the system comprising:
a. a first body engaging assembly for engaging first body portions of a user adjacent a joint of the body;
b. a second body engaging assembly for engaging second body portions of the user; and
c. a positioning assembly that rotatably positions the first body engaging assembly relative to the second body engaging assembly for static progressive positioning of the joint in pronation or supination, the positioning assembly comprising:
i. a ring member, the first body engaging member being mounted adjacent an interior portion of the ring member,
ii. a yoke to which the ring member is movably mounted,
iii. and a rotator mounted to the yoke and positioned to rotationally and frictionally engage the ring member to move the ring member for positioning of the joint.

2. The system of claim 1, wherein the first body engaging assembly is configured for engaging a hand and a wrist of the user.

3. The system of claim 1, wherein the first body engaging assembly comprises a first receiver configured to receive a hand of the user and a second receiver configured for receiving a wrist of the user.

4. The system of claim 1, wherein the second body engaging assembly is configured for engaging an elbow and portions of a bicep and forearm of the user adjacent the elbow.

5. The system of claim 1, wherein the second body engaging assembly comprises a substantially L-shaped receiver configured for receiving an elbow and portions of a bicep and a forearm of the user adjacent the elbow.

6. The system of claim 1, wherein the ring member has a partial ring configuration.

7. The system of claim 1, wherein the ring member has a substantially circular cross-section.

8. The system of claim 1, wherein the rotator frictionally engages a portion of an outer periphery of the ring member.

9. The system of claim 1, wherein the positioning assembly further comprises a stop operable to prevent movement of the ring member once the ring member has been moved to a desired position.

10. A patient care system for providing static progressive positioning of body members in pronation or supination, the system comprising:
a rotatable ring member;
a rotator operably associated with the ring member to rotate the ring member for positioning of the joint
a first body engaging assembly mounted on the ring member and configured for engaging first body portions of a user adjacent a joint of the body;
a second body engaging assembly remote from the first body engaging member and configured for engaging second body portions of the user;
wherein operation of the rotator causes rotation of the ring member to rotatably position the first body engaging assembly relative to the second body engaging assembly for static progressive positioning of the joint in pronation or supination.

11. The patient care system of claim 10, wherein the ring member has a partial ring configuration.

12. The system of claim 1, wherein the ring member has a substantially circular cross-section.

13. The patient care system of claim 10, wherein the first body engaging assembly is configured for engaging a hand and a wrist of the user.

14. The system of claim 10, wherein the second body engaging assembly comprises a substantially L-shaped receiver configured for receiving an elbow and portions of a bicep and a forearm of the user adjacent the elbow.

15. The system of claim 10, wherein the rotator comprises a shaft having a knob at one end thereof for manipulation by a user to rotate the rotator and a rubber cylinder located on the shaft for frictionally engaging a portion of an outer periphery of the ring member so that rotation of the knob results in rotation of the ring member.
